(19) United States
(12) Patent Application Publication
(10) Pub. No.: US 2026/0128143 A1
(43) Pub. Date: May 7, 2026

(54) METHOD OF PROVIDING GOAL ORIENTATED GUIDANCE

(71) Applicant: David Boyd Murray, JR., Mountoursville, PA (US)

(72) Inventor: David Boyd Murray, JR., Mountoursville, PA (US)

(21) Appl. No.: 19/370,074

(22) Filed: Oct. 27, 2025

Related U.S. Application Data
(60) Provisional application No. 63/716,352, filed on Nov. 5, 2024.

(51) Int. Cl. G16H 20/00; G06Q 10/0631; G06Q 10/1093; G16H 10/20

(52) U.S. Cl. CPC G16H 20/00; G06Q 10/063112; G06Q 10/1093; G16H 10/20

(57) ABSTRACT

A method of providing goal orientated guidance that includes interaction with a mentor. The method can be used with person to person interaction, software applications (APP), Artificial Intelligence software (AI) or a combination of all three. A participant desires to improve an area of the participant's life and initiates contact with the service provider. There is an intake of the participant's data. Included in the intake of data are at least three evaluation surveys.

This application claims the benefit of and incorporates by reference U.S. Provisional Application No.: 63/716,352, filed Nov. 5, 2024.

### BACKGROUND

The field of invention generally relates to a method of providing goal orientated guidance. More specifically, the present invention relates to a method of providing goal orientated guidance with mentor interaction.

Currently, there are few structured methods for helping a user develop and execute a plan to meet the goals of the user based on the strengths and weaknesses of the user. Most available guidance is found in self help books or information found on the internet. It is an object of the present invention to provide method that can be used in person or through a software application to guide a user with a plan to meet the goals of a user based on the strengths and weaknesses of the user.

### SUMMARY OF THE INVENTION

A method of providing goal orientated guidance that includes interaction with a mentor. The method can be used with person to person interaction, software applications (APP), Artificial Intelligence software (AI) or a combination of all three. A participant desires to improve an area of the participant's life and initiates contact with the service provider. There is an intake of the participant's data. Included in the intake of data are at least three evaluation surveys.

### BRIEF DESCRIPTION OF DRAWINGS

FIG. 1 is a schematic flow chart of a method according to the present invention.

FIG. 2 is a schematic flow chart of a method according to the present invention.

FIG. 3 is schematic flow chart of a method according to the present invention.

FIG. 4 is a schematic flow chart of a method according to the present invention.

FIG. 5 is a schematic flow chart of a method according to the present invention.

FIG. 6 is a schematic flow chart of a method according to the present invention.

FIG. 7 is a schematic flow chart of a method according to the present invention.

FIG. 8 is a schematic flow chart of a method according to the present invention.

FIG. 9 is a schematic flow chart of a method according to the present invention.

### DETAILED DESCRIPTION

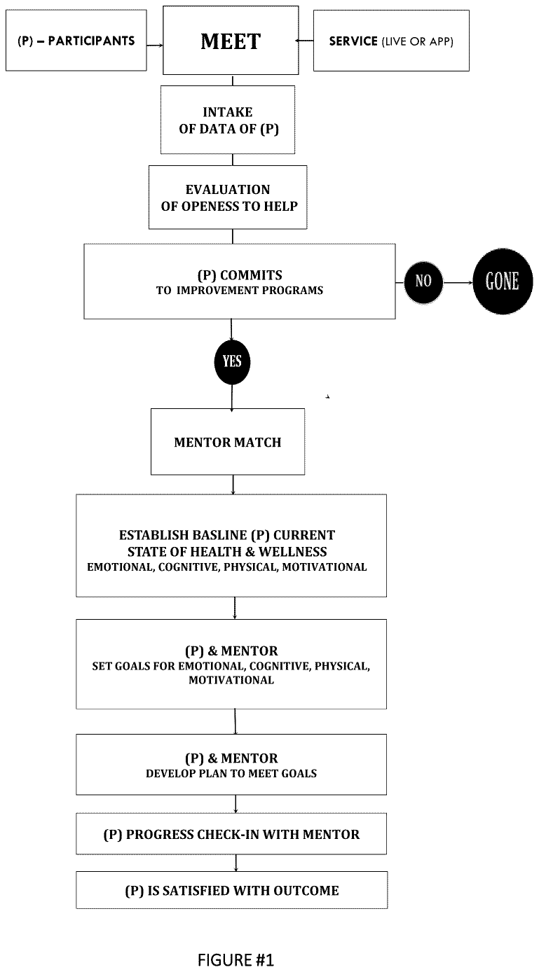

The present invention is a method of providing goal orientated guidance that includes interaction with a mentor. The method can be used with person to person interaction, software applications (APP), Artificial Intelligence software (AI) or a combination of all three. FIG. 1 shows an outline flow chart of the method. A user is labeled as a participant (P). In the first step, the participant initiates contact with a guidance service provider. The participant meets in a person to person meeting or begins contact with the APP during the first step of initial contact.

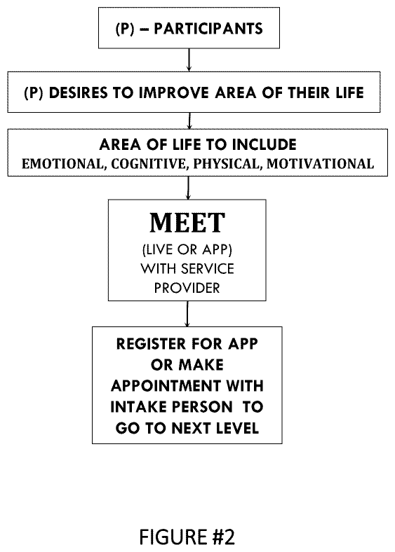

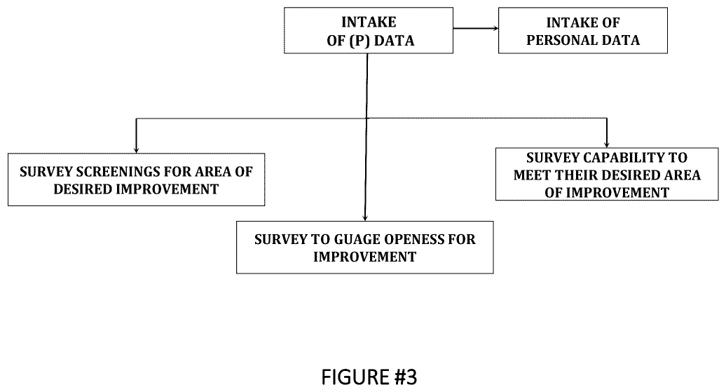

The method using the person to person contact initially will be described as the first embodiment. The second embodiment will employ the APP and or AI using the same basic method. The participant desires to improve an area of the participant's life and initiates contact with the service provider to make an appointment with an intake person, as shown in FIG. 2. FIG. 1 shows the next step is to receive an intake of the participant's data by the intake person. The intake of data is shown in FIG. 3. The intake of data includes the recording of the participant's personal data. The personal data can include name, address and contact information. The personal data can also include physical characteristics data, such as height, weight, sex and other such type of data. Also included in the intake of data are three evaluation surveys, which are a desired improvement survey, an openness survey and a capability survey, as shown in FIG. 3.

A desired improvement survey is made up of questions to screen for areas of improvement desired by the participant. The desired area of improvement can include improvements to mental state, mental performance, academic performance, physical performance, motivational help or other interest areas. Most improvements fall under four major categories of the participant's state of being, which are Emotional, Cognitive, Physical and Motivational. Examples of a desired improvement on interest areas are improving on academic skills, athletic skills, dealing with emotional setbacks, as well as other improvements that fall under the four major categories. A desired improvement can include guidance with one or more of the major categories being involved and one or more areas of interest involved. The openness survey includes questions to gauge the openness of the participant to make the improvement desired. Openness relates to possibilities the may hold the participant back from achieving the goal of the participant. Examples of these possibilities include fear of what is required to meet the goal, fear of failure, stubbornness/wiliness of the participant to perform needed steps of a plan to meet the goal, etc. The capability survey includes questions of whether the participant has any restraints of achieving the goal of the improvement due to the capabilities of the participant. The survey identifies if the participant's abilities will not allow the participant to meet the goal of the participant. For example, a goal of growing another foot in height may not be achievable if the participant is fully grown. The capability survey will based on the interest areas of the desired goal of the participant.

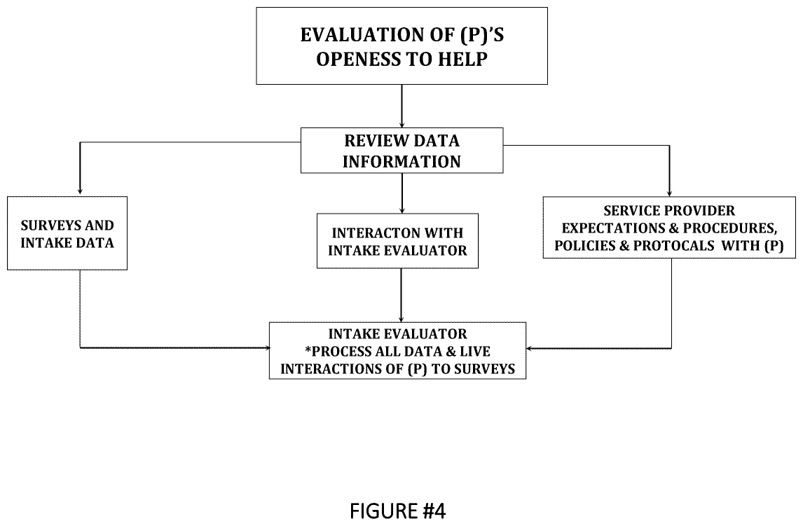

FIGS. 1 and 4 show that the next step after of intake of data is the evaluation of openness to receive the necessary guidance. FIG. 4 shows the reviewing of data information from the intake. The data information included information from the intake data and surveys, interaction notes of the intake evaluator and alignment with service provider expectations and procedures. Procedures include policies and safeguards of the service provider. The intake evaluator then processes all data and live interactions with the participant to determine if the participant is open to receive the necessary guidance. If the answer is yes, the participant moves to the next step. If the answer is no, the participant may choose to reevaluate the goal or choose not enter in the program provided by the service provider.

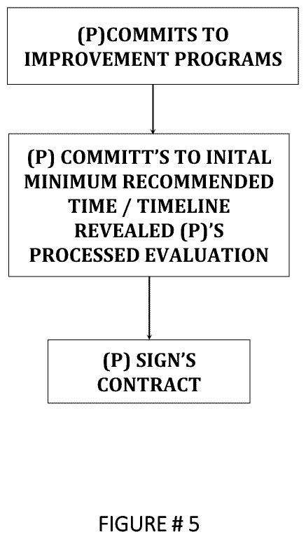

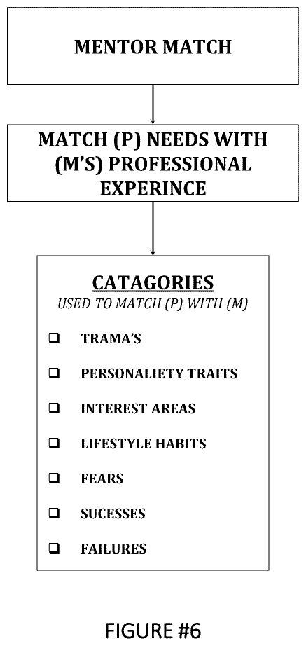

The next step is to see if the participant is open to the necessary guidance, as shown in FIG. 1 and FIG. 5. The participant commits to an initial minimum of involvement based on an initial recommendation of time commitment and a time line. The processed evaluation of the participant is shared with the participant during this step. The participant must then engage into a contract with the service provider to commit the time and resources to the time commitment and time line based on the processed evaluation of the participant. Once the participant has made the commitment, the participant is matched with a mentor, as shown in FIGS. 1 and 6. In the step of the mentor match, the needs of the participant are matched with the professional experience and categories of experience of available mentors. The mentors provide personal interaction with the participant. Some the categories include traumas, personality traits, interest areas, lifestyle habits, fears successes and failures. Include in the mentor matching is consideration if the mentor has shared life experiences that align with the life experiences of the participant.

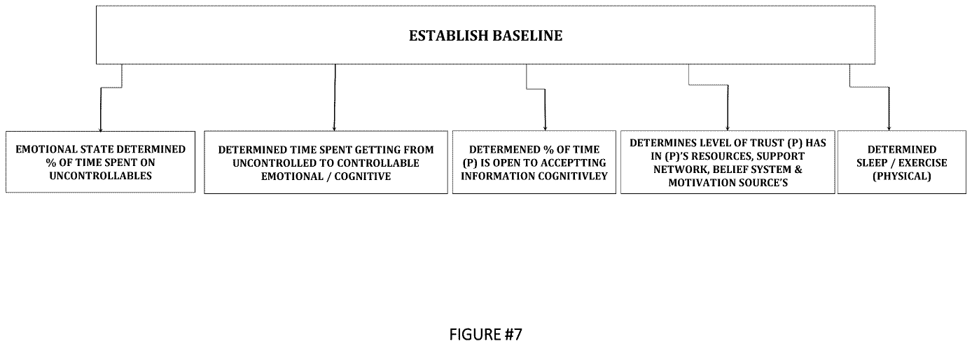

FIG. 1 and FIG. 7 show the next step is establishing a baseline of the current state of health and wellness that includes the four major categories of Emotional state, Cognitive state, Physical State and Motivational State. Establishing a baseline will aid the interaction with the mentor and the participant to achieve the goal of the participant. The mentor and participant will determine the percentage of time the participant usually spends on uncontrollable situations encounter by the participant, which falls under the emotional state. The percentage of time the participant spends on uncontrollable situations relates to whether the participant is more likely or less likely to spend time on situations or issues the participant cannot control. The mentor and participant will determine the time spent by the participant on uncontrollable issues verses controllable issues, which falls under the emotional and cognitive states. The amount time spent by the participant on uncontrollable issue verses issues that are controllable by the participant can be valuable information. The mentor and participant will determine the percentage of time the participant is open to accept information, which falls under the cognitive state. Percentage of time the participant is open to accept information relates to whether the participant is more often or less often in a state of mind to accept information. The mentor and participant will determine how much the participant trusts, any belief systems used by the participant and does the participant have a support network, which all falls under motivation states. Determining the ability to trust, the belief systems of the participant and the support network of the participant will help the mentor work with the participant. The mentor and participant will determine sleep, exercise and other physical factors that surround and affect the life of the participant, which all falls under the physical state and the cognitive state. Determining the sleep, nutrition, exercise and other physical factors of the participant allows the service provider to determine the physical state of the participant. The physical state may show the participant may have limits to make desired improvements to meet the goals of the participant and if the physical state may be improved. For instance, malnutrition would limit the participant in many ways, but is a condition that can be easily improved with the correct motivation.

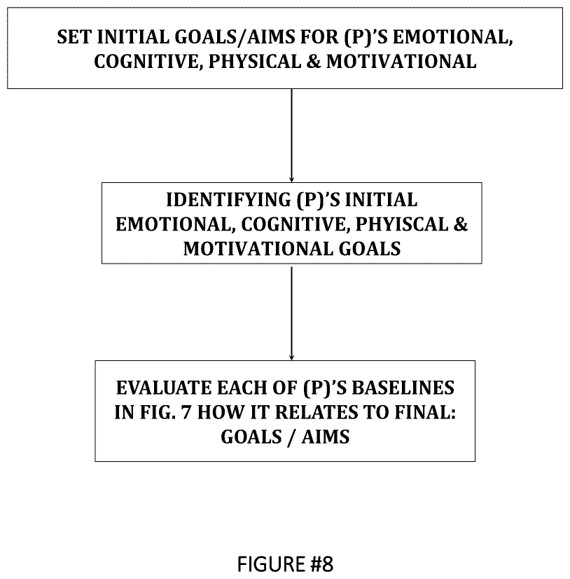

FIG. 1 and FIG. 8 show the next step where the participant and the mentor set the goal or goals of the participant. When setting the goal of the participant, the participant and the mentor take in account the established baseline and the four major categories of Emotional state, Cognitive state, Physical State and Motivational State. The participant and the mentor identify the final Emotional state, Cognitive state, Physical State and Motivational State improvements to meet the goal of the participant. This is done by evaluating the established baseline and how it relates to the goal and aims of the participant.

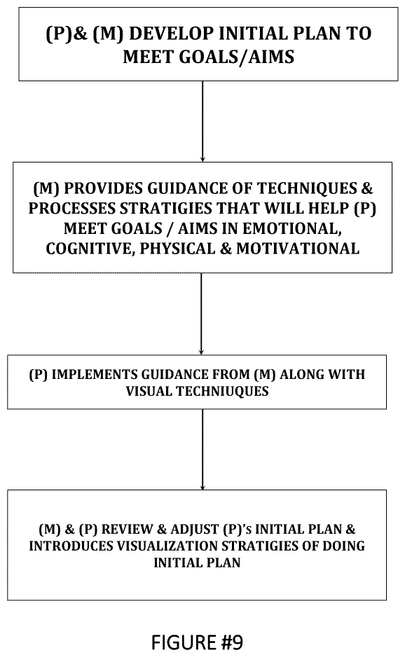

FIG. 1 and FIG. 9 show the next step is where the participant and the mentor develop a plan to meet the goal and aims of the participant. The mentor will provide guidance of techniques and process strategies developed by the service provider that will help the participant meet the goal and aims of participant. This guidance will include techniques and process strategies including one or more of the four major categories of Emotional state, Cognitive state, Physical State and Motivational State. The participant and the mentor will review and make adjustments to formulate an initial plan. The initial plan will include the introduction of visualizations strategies for implementing the initial plan. From there, the participant will implement the initial plan and have scheduled progress check-in reviews with the mentor, as shown in FIG. 1. When the participant feels comfortable that the goal has been met, the participant may choose to end working with the service provider.

The second embodiment will include using the method as part of a software application (APP), along with Artificial Intelligence software (AI). Where the APP can be used on a computer or mobile device, such as a cell phone or tablet. The APP will function as a data input device, but is not limited to that function. The AI will simulate human interaction with the participant, but is not limited to that function. The simulation of human interactions can include the back and forth between the participant and the mentor. AI will be able to interpret responses from the participant and provide real time guidance similar to human, including planning next steps. The participant desires to improve an area of the participant's life and initiates contact with the service provider by an APP, typically on the computer or phone of the participant to provide intake data. This can include making an appointment with an intake person, as shown in FIG. 2, or filling out a survey using the APP. AI can be used interact with the participant for the survey. FIG. 1 shows the next step is to receive an intake of the participant's data by the APP. The intake of data is shown in FIG. 3. The intake of data includes the recording of the participant's personal data. The personal data can include name, address and contact information. The personal data can also include physical characteristics data, such as height, weight, sex and other such type of data. Also included in the intake of data are three evaluation surveys, which are a desired improvement survey, an openness survey and a capability survey, as shown in FIG. 3. The surveys can be interactive though the APP, including the use of AI to provide real time feedback to the participant.

A desired improvement survey is made up of questions to screen for areas of improvement desired by the participant. The desired area of improvement can include improvements to mental state, mental performance, academic performance, physical performance, motivational help or other interest areas. Most improvements fall under four major categories of the participant's state of being, which are Emotional, Cognitive, Physical and Motivational. Examples of a desired improvement on interest areas are improving on academic skills, athletic skills, dealing with emotional setbacks, as well as other improvements that fall under the four major categories. A desired improvement can include guidance with one or more of the major categories being involved and one or more areas of interest involved. The openness survey includes questions to gauge the openness of the participant to make the improvement desired. Openness relates to possibilities the may hold the participant back from achieving the goal of the participant. Examples of these possibilities include fear of what is required to meet the goal, fear of failure, stubbornness/wiliness of the participant to perform needed steps of a plan to meet the goal, etc. The capability survey includes questions of whether the participant has any restraints of achieving the goal of the improvement due to the capabilities of the participant. The survey identifies if the participant's abilities will not allow the participant to meet the goal of the participant. For example, a goal of growing another foot in height may not be achievable if the participant is fully grown. The capability survey will based on the interest areas of the desired goal of the participant.

FIGS. 1 and 4 show that the next step after of intake of data is the evaluation of openness to receive the necessary guidance. FIG. 4 shows the reviewing of data information from the intake by the APP. The data information included information from the intake data and surveys, interaction notes of the intake evaluator and alignment with service provider expectations and procedures. Procedures include policies and safeguards of the service provider. The intake evaluator can be replaced by the APP and/or AI to processes all data and interactions of the participant to determine if the participant is open to receive the necessary guidance. AI can be employed to enhance the evaluation. If the answer is yes, the participant moves to the next step. If the answer is no, the participant may choose to reevaluate the goal or choose not enter in the program provided by the service provider.

The next step is to see if the participant is open to the necessary guidance, as shown in FIG. 1 and FIG. 5. The participant commits to an initial minimum of involvement based on an initial recommendation of time commitment and a time line. The initial minimum of involvement is calculated using the APP. AI can be used to provide interaction with the participant during the calculation on the minimum involvement.

The processed evaluation of the participant is shared with the participant during this step. The participant must then engage into a contract with the service provider to commit the time and resources to the time commitment and time line based on the processed evaluation of the participant. The engagement can be performed through the APP. Once the participant has made the commitment, the participant is matched with a live mentor or AI equivalent, as shown in FIGS. 1 and 6. In the step of the mentor match, the needs of the participant are matched with the professional experience and categories of experience of available mentors. The mentors provide personal interaction with the participant. Some the categories include traumas, personality traits, interest areas, lifestyle habits, fears successes and failures. Include in the mentor matching is consideration if the mentor has shared life experiences that align with the life experiences of the participant. The mentor can be replaced by AI generated virtual mentor through the APP to perform the same tasks. IF AI is not used, the live mentor can use video conferencing through the APP.

FIG. 1 and FIG. 7 show the next step is establishing a baseline of the current state of health and wellness that includes the four major categories of Emotional state, Cognitive state, Physical State and Motivational State. Establishing a baseline will aid the interaction with the mentor/APP/AI and the participant to achieve the goal of the participant. The mentor/APP/AI and participant will determine the percentage of time the participant usually spends on uncontrollable situations encounter by the participant, which falls under the emotional state. The percentage of time the participant spends on uncontrollable situations relates to whether the participant is more likely or less likely to spend time on situations or issues the participant cannot control. The mentor/APP/AI and participant will determine the time spent by the participant on uncontrollable issues verses controllable issues, which falls under the emotional and cognitive states. The amount time spent by the participant on uncontrollable issue verses issues that are controllable by the participant can be valuable information. The mentor/APP/AI and participant will determine the percentage of time the participant is open to accept information, which falls under the cognitive state. Percentage of time the participant is open to accept information relates to whether the participant is more often or less often in a state of mind to accept information. The mentor/APP/AI and participant will determine how much the participant trusts, any belief systems used by the participant and does the participant have a support network, which all falls under motivation states. Determining the ability to trust, the belief systems of the participant and the support network of the participant will help the mentor work with the participant. The mentor/APP/AI and participant will determine sleep, exercise and other physical factors that surround and affect the life of the participant, which all falls under the physical state and the cognitive state. Determining the sleep, nutrition, exercise and other physical factors of the participant allows the service provider to determine the physical state of the participant. The physical state may show the participant may have limits to make desired improvements to meet the goals of the participant and if the physical state may be improved. For instance, malnutrition would limit the participant in many ways, but is a condition that can be easily improved with the correct motivation. This baseline can be done through the APP using video conferencing or using an AI generated mentor through the APP.

FIG. 1 and FIG. 8 show the next step where the participant and the mentor/APP/AI set the goal or goals of the participant. When setting the goal of the participant, the participant and the mentor take in account the established baseline and the four major categories of Emotional state, Cognitive state, Physical State and Motivational State. The participant and the mentor identify the final Emotional state, Cognitive state, Physical State and Motivational State improvements to meet the goal of the participant. This is done by evaluating the established baseline and how it relates to the goal and aims of the participant. The setting of the goal can be done through the APP using video conferencing or using an AI generated mentor through the APP.

FIG. 1 and FIG. 9 show the next step is where the participant and the mentor/APP/AI develop a plan to meet the goal and aims of the participant. The mentor/APP/AI will provide guidance of techniques and process strategies developed by the service provider that will help the participant meet the goal and aims of participant. This guidance will include techniques and process strategies including one or more of the four major categories of Emotional state, Cognitive state, Physical State and Motivational State. The participant and the mentor/APP/AI will review and make adjustments to formulate an initial plan. The initial plan will include the introduction of visualizations strategies for implementing the initial plan. The plan can be developed through the APP using video conferencing or using an AI generated mentor through the APP From there, the participant will implement the initial plan and have scheduled progress check-in reviews with the mentor or the APP, as shown in FIG. 1. When the participant feels comfortable that the goal has been met, the participant may choose to end working with the service provider.

I claim:

1. A method of providing goal orientated guidance comprising the steps of:
initiating contact with a guidance service provider by a participant, where the participant desires to improve an area of the participant's life and initiates contact with the service provider to make an appointment with an intake person;
receiving an intake of the participant's data by the intake person, where the intake of data includes the recording of the participant's personal data, where the personal data can include name, address and contact information, where the personal data can include physical characteristics data of the participant, where the intake of data includes at least three evaluation surveys, the first survey being a desired improvement survey made up of questions to screen for areas of improvement desired by the participant, desired area of improvement survey can include improvements to mental state, mental performance, academic performance, physical performance, motivational help and other interest areas, where improvements fall under four major categories of the participant's state of being which are Emotional, Cognitive, Physical and Motivational, the second survey being an openness survey that includes questions to gauge the openness of the participant to make the improvement desired, where openness relates to possibilities the may hold the participant back from achieving the goal of the participant, the third capability survey including questions of whether the participant has any restraints of achieving the goal of the improvement due to the capabilities of the participant, the capability survey identifies if the participant's abilities will not allow the participant to meet the goal of the participant;
reviewing of data information from the intake, the data information includes information from the intake data and surveys, interaction notes of the intake evaluator and alignment of the goal with service provider expectations, procedures, policies and safeguards;
processing all data and live interactions with the participant to determine if the participant is open to receive guidance, if the answer is yes, the participant moves to the next step and if the answer is no, the participant may choose to reevaluate the goal or choose not enter in the program provided by the service provider;
committing to an initial minimum of involvement by the participant based on an initial recommendation of time commitment and a time line, where the processed evaluation of the participant is shared with the participant;
the participant engages into a contract with the service provider to commit the time and resources to the time commitment and time line based on the processed evaluation of the participant;
matching of the participant with a mentor to provide personal interaction with the participant, where the needs of the participant are matched with the professional experience and categories of experience of available mentors, where the categories of experience can include traumas, personality traits, interest areas, lifestyle habits, fears successes and failures, and where mentor matching considers if the mentor has shared life experiences that align with the life experiences of the participant;
establishing a baseline of the current state of health and wellness that includes the four major categories of Emotional state, Cognitive state, Physical State and Motivational State, where establishing a baseline will aid the interaction with the mentor and the participant to achieve the goal of the participant;
determining by the mentor and participant the percentage of time the participant usually spends on uncontrollable situations encounter by the participant, which falls under the emotional state, where the percentage of time the participant spends on uncontrollable situations relates to whether the participant is more likely or less likely to spend time on situations and issues the participant cannot control;
determining by the mentor and participant the time spent by the participant on uncontrollable issues verses controllable issues, which falls under the emotional and cognitive states, where the amount time spent by the participant on uncontrollable issues verses issues that are controllable by the participant can be valuable information;
determining by the mentor and participant the percentage of time the participant is open to accept information, which falls under the cognitive state, where the percentage of time the participant is open to accept information relates to whether the participant is more often or less often in a state of mind to accept information;
determining by the mentor and participant how much the participant trusts any type of belief systems used by the participant and does the participant have a support network, which all falls under motivation states, where determining the ability to trust the belief systems of the participant and the support network of the participant will help the mentor work with the participant;
determining by the mentor and participant sleep, exercise and other physical factors that surround and affect the life of the participant, which all falls under the physical state and the cognitive state, where determining the sleep, nutrition, exercise and other physical factors of the participant allows the service provider to determine the physical state of the participant, where the physical state may show the participant may have limits to make desired improvements to meet the goals of the participant and if the physical state may be improved;
setting of the goal or goals of the participant by the participant and the mentor, where setting the goal of the participant, the participant and the mentor take in account the established baseline and the four major categories of Emotional state, Cognitive state, Physical State and Motivational State. The participant and the mentor identify the final Emotional state, Cognitive state, Physical State and Motivational State improvements to meet the goal of the participant. This is done by evaluating the established baseline and how it relates to the goal and aims of the participant.
developing by the participant and the mentor of a plan to meet the goal and aims of the participant, where the mentor provides guidance of techniques and process strategies developed by the service provider that will help the participant meet the goal and aims, where the guidance includes techniques and process strategies that include one or more of the four major categories of Emotional state, Cognitive state, Physical State and Motivational State;
reviewing and making adjustments by the participant and the mentor to formulate an initial plan, where the initial plan will include the introduction of visualizations strategies for implementing the initial plan; and
implementing by the participant of the initial plan and scheduling progress check-in reviews with the mentor, where when the participant feels comfortable that the goal has been met, the participant may choose to end working with the service provider.

2. The method of claim 1, wherein the participant initiates contact with the service provider using an APP, typically on the computer or mobile device phone of the participant to provide intake data.

3. The method of claim 1, wherein the participant makes an appointment with an intake person using an APP.

4. The method of claim 1, wherein the participant interacts with AI to provide the participant's data.

5. The method of claim 1, wherein responses by the participant to the surveys is done using an APP.

6. The method of claim 5, wherein interactions between the participant and the APP is done using AI.

7. The method of claim 1, wherein AI evaluates the openness of the participant to receive the necessary guidance.

8. The method of claim 1 wherein the intake evaluator is replaced by AI to processes all data and interactions of the participant to determine if the participant is open to receive the necessary guidance.

9. The method of claim 1, wherein minimum of involvement is calculated using the APP.

10. The method of claim 1, wherein once the participant has made the commitment, the participant is matched with AI equivalent of a human mentor.

11. The method of claim 1, wherein establishing a baseline of the current state of health and wellness using the APP.

12. The method of claim 11, wherein AI is used to for establishing a baseline.

13. The method of claim 1, wherein the APP and participant determine the percentage of time the participant usually spends on uncontrollable situations encounter by the participant, the time spent by the participant on uncontrollable issues verses controllable issues, determine the percentage of time the participant is open to accept information, determine how much the participant trusts any belief systems used, determine sleep, exercise and other physical factors that surround and affect the life of the participant.

14. The method of claim 13, wherein AI is used to do the determinations listed to provide human feedback.

15. The method of claim 1, wherein the participant uses the APP to set the goal of the participant.

16. The method of claim 15, wherein AI is used to interact with the participant to set the goal of the participant.

17. The method of claim 1, wherein the participant uses the APP to develop a plan to meet the goal and aims of the participant, including guidance of techniques and process strategies that will help the participant meet the goal and aims of participant.

18. The method of claim 17, wherein AI is used to form the plan to provide human feedback.

19. The method of claim 1, wherein the participant and the APP reviews and make adjustments to formulate an initial plan, where the initial plan includes the introduction of visualizations strategies for implementing the initial plan.

20. The method of claim 19, wherein AI is used to form the plan to provide human feedback.
